(19) United States
(12) Plant Patent Application Publication
(10) Pub. No.: US 2016/0360689 P1
(43) Pub. Date: Dec. 8, 2016

(54) Anthurium plant named 'ANTHDOSDOH'

(50) ;

(71) Applicant: ANTHURA B.V., Bleiswijk (NL)

(72) Inventor: Jan Van Dijk, Bleiswijk (NL)

(21) Appl. No.: 14/545,658

(22) Filed: Jun. 3, 2015

(51) Int. Cl. A01H 5/00

(52) U.S. Cl. PLT/367

(57) ABSTRACT

A new and distinct variety of Anthurium plant named ‘ANTHDOSDOH’ particularly characterized by having a pink, lanceolate spathe, a purple spadix with a red tip, a lanceolate leaf, an extremely compact plant habit and light green foliage is disclosed.

### GENUS AND SPECIES

Anthurium andreanum L.

### VARIETY DENOMINATION

‘ANTHDOSDOH’

### BACKGROUND OF THE NEW PLANT

The present invention comprises a new and distinct variety of Anthurium, botanically known as Anthurium andreanum L., and hereinafter referred to by the variety name ‘ANTHDOSDOH’. The new variety originated from a hybridization made in September 2006 in Bleiswijk, The Netherlands. The female parent is a light pink Anthurium pot plant designated ‘9941-01’ (unpatented), and the male parent was a pink Anthurium plant designated ‘9741-04’ (unpatented).

A single plant was selected in June 2008 and has been asexually reproduced repeatedly by tissue culture in Bleiswijk, The Netherlands over a seven-year period. The present invention has been found to retain its distinctive characteristics through successive asexual propagations.

Plant Breeder's Rights for this variety have been applied for in the European Union on Nov. 19, 2012, in Japan on May 26, 2014 and in Vietnam on Nov. 26, 2014. ‘ANTHDOSDOH’ has not been made publicly available or sold anywhere in the world more than one year prior to the filing of this application.

### SUMMARY OF THE INVENTION

The following are the most outstanding and distinguishing characteristics of this new variety when grown under normal horticultural practices in Bleiswijk, The Netherlands:
1) Pink, lanceolate spathe;
2) Purple spadix with red tip;
3) Lanceolate leaf;
4) Extremely compact plant habit; and
5) Light green foliage.

### DESCRIPTION OF THE PHOTOGRAPHS

This new Anthurium plant is illustrated by the accompanying photographs which show the overall plant habit including blooms and foliage of the plant; the colors shown are as true as can be reasonably obtained by conventional photographic procedures. The photographs are of a 50-week old plant grown in a greenhouse in Bleiswijk, The Netherlands in April 2015.

FIG. 1 shows the overall plant habit, including blooms and foliage.

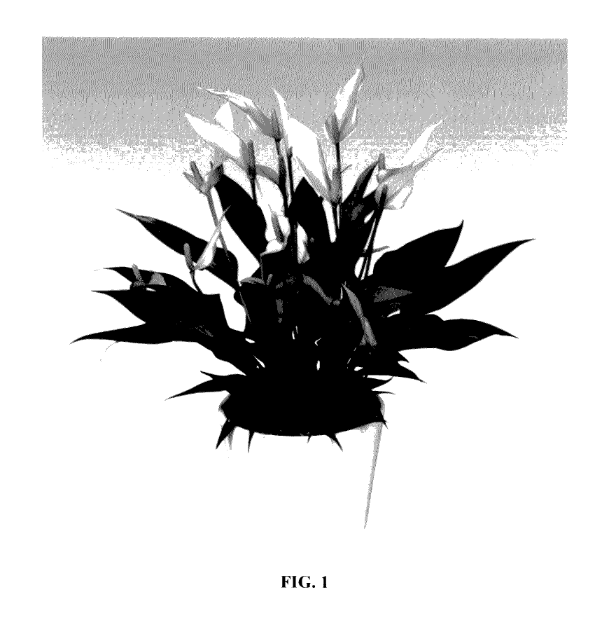

FIG. 2 shows a close-up of the mature spathe.

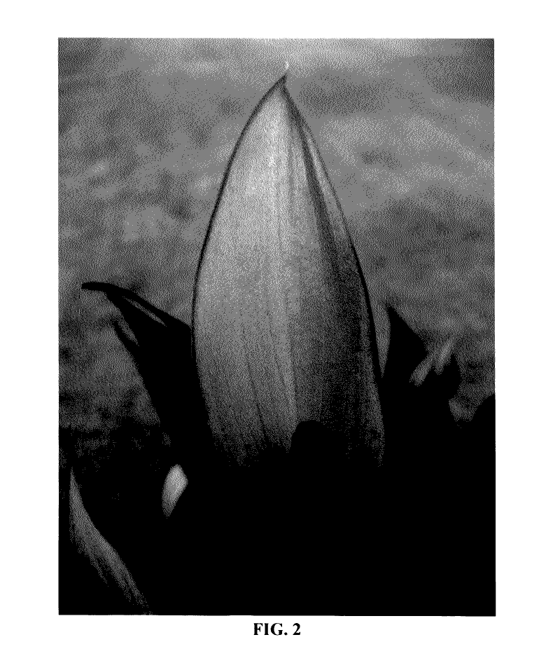

FIG. 3 shows the upper leaf blade surface.

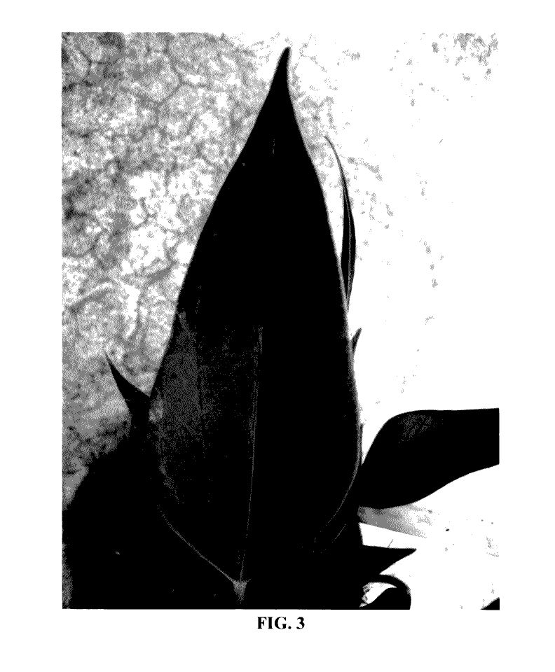

### DESCRIPTION OF THE NEW VARIETY

The following detailed description sets forth the distinctive characteristics of ‘ANTHDOSDOH’. The data which define these characteristics were collected from asexual reproductions carried out in Bleiswijk, The Netherlands. The plant history was taken on 50-week old plants which were planted from tissue culture in 12-centimeter pots and grown in a glass greenhouse between 19° C. and 24° C. Observations were made in April 2015. Color readings were taken under 5000 lux natural light in the greenhouse. Color references are primarily to The R.H.S. Colour Chart of The Royal Horticultural Society of London (R.H.S.) (2001).

### DETAILED BOTANICAL DESCRIPTION

Classification:
   Family.—Araceae.
   Botanical.—Anthurium andreanum L.
   Common name.—Anthurium.
   Demonimation.—‘ANTHDOSDOH’.
Parentage:
   Female parent.—Anthurium plant ‘9941-01’ (unpatented).
   Male parent.—Anthurium plant ‘9741-04’ (unpatented).
Plant:
   Propagation.—Tissue culture.
   Root description.—Fleshy white-cream colored roots with small hairy lateral roots having yellow-colored root tips.
   Time to produce a finished flowering plant.—25 to 30 weeks for a 12 cm pot.
   Growth habit.—Herbaceous perennial.
   Height (measured from soil, including inflorescence).—20.0 cm to 24.0 cm.
   Width (measured from leaf tips).—25.0 cm to 30.0 cm.
Leaves:
   Immature leaves.—Length: 5.0 cm to 9.0 cm Width: 2.0 cm to 4.0 cm Color: Upper surface: RHS 144A Lower surface: RHS 144B Texture (both upper and lower surfaces): Shiny.
   Mature leaves.—Length (fully expanded): 9.0 cm to 13.0 cm Width: 3.0 cm to 5.0 cm Shape: Lanceolate Apex: Acuminate Base: Cuneate Leaf blade angle with the petiole: Between 140 degrees and 160 degrees Leaf margin: Entire Color: Upper surface: RHS 137A Lower surface: RHS 146B Texture: Shiny, leathery and thick Venation: Pinnate veining; the mid-vein and primary veins (the veins that radiate out from the junction of petiole and leaf) protrude at the underside of the leaf blade Venation color: Upper surface: RHS 146B Lower surface: RHS 146C.
   Lobes.—Not present.
   Petiole.—Cross-section: Round Diameter: 0.2 cm to 0.3 cm Length: 4.0 cm to 8.0 cm for a mature leaf size Color: Mature leaf: RHS 146C Immature leaf: RHS 146C Cataphyll color surrounding the petiole: Outside: RHS 144A Inside: RHS 144A.
   Geniculum.—Length: 0.8 cm to 1.0 cm Width: 0.3 cm to 0.4 cm Color: RHS 146C.
Inflorescence:
   Arrangement.—Single.
   Flowering habit (length of flowering season).—Continuous.
   Number of inflorescences per plant.—7 to 10.
   Fragrance.—Absent.
   Longevity of inflorescence on plant.—Over a year.
Spathe:
   Buds.—The spathe is tightly rolled around the spadix and extrudes from the peduncle sheath. After the spathe is fully open the peduncle elongates some extra centimeters.
   Arrangement.—Spathe angle with the peduncle is between 130 degrees and 150 degrees; the spathe stands on a wiry peduncle about 5.0 cm to 7.0 cm above the foliage.
   Shape.—Lanceolate.
   Apex.—Acuminate.
   Base.—Rotundate.
   Texture.—Shiny and smooth.
   Margin.—Entire.
   Size.—Length: 6.0 cm to 7.0 cm Width: 2.0 cm to 3.0 cm.
   Color.—Just fully open: Upper surface: RHS 54A Lower surface: RHS 54C After opening, the spathe slowly starts fading from pink to light pink.
Peduncle:
   Shape.—Erect.
   Cross-section.—Round.
   Length.—12.0 cm to 15.0 cm.
   Diameter.—0.2 cm to 0.3 cm.
   Color.—RHS 152B.
Flowering time:
   General.—One small rooted untreated tissue culture plant of 2.0 cm tall will flower after 3 to 4 months, depending on the season, and 4 to 6 blossoms will appear. More blossoms appear after some additional weeks so that a full flowering and commercial plant will have 7 to 10 pink spathes. Smaller blossoms may occur on less mature plants.
Spadix:
   Size.—Length: 1.5 cm to 2.5 cm (depending on flower size) Width (at apex): 0.35 cm to 0.45 cm Width (at base): 0.4 cm to 0.5 cm.
   Shape.—Columnar.
   Angle from spadix tip to peduncle.—160 to 180 degrees (spadix is almost in one line with the peduncle).
   Texture.—When the spathe is unfurling the spadix is smooth. When the spadix matures, small stigmata protrude. The stigmata are evenly distributed round the spadix. The spadix matures from base to top, slowly giving the spadix a somewhat rough appearance.
   Color.—Immature: RHS 47A Mature: RHS 61A.
Flowers:
   Quantity per spadix.—90 to 110.
   Spadix flower arrangement.—Bisexual, rounded in cross-section.
   Shape.—Rounded.
   Size.—Length: 0.05 cm to 0.1 cm Diameter (maximum): 0.1 cm.
   Color.—RHS 61A.
Reproductive organs:
   Stamens.—Not visible.
   Pollen amount.—Very few.
   Pollen color.—RHS 156D.
   Pistil.—Quantity: Many Length: Less than 0.01 cm Color: RHS 61C.
   Style.—Not observed.
   Stigma.—Shape: Ovoid Diameter: Less than 0.01 cm Color: RHS 61C.
   Ovary.—Rarely visible.
   Ovary color.—Not measured.
Fruit and seed set: None observed

### COMPARISON WITH PARENTAL AND COMMERCIAL VARIETIES

‘ANTHDOSDOH’ differs from the female parent plant ‘9941-01’ (unpatented) in that ‘ANTHDOSDOH’ has a pink spathe, whereas ‘9941-01’ has a light pink spathe.

‘ANTHDOSDOH’ differs from male parent plant ‘9741-04’ (unpatented) in that ‘ANTHDOSDOH’ has a lanceolate spathe, whereas ‘9741-04’ has an ovate spathe.

‘ANTHDOSDOH’ differs from commercial variety ‘ANTHUCOEN’ (U.S. Plant Pat. No. 22,630) in that ‘ANTHDOSDOH’ has a small, lanceolate spathe, whereas ‘ANTHUCOEN’ has a larger, more ovate spathe. Additionally, ‘ANTHDOSDOH’ has lanceolate leaves, whereas ‘ANTHUCOEN’ has cordate leaves.

‘ANTHDOSDOH’ differs from commercial variety ‘ANTHEBENEX’ (unpatented) in that ‘ANTHDOSDOH’ has a pink spathe, whereas ‘ANTHEBENEX’ has a purple spathe. Additionally, ‘ANTHDOSDOH’ has small, lanceolate leaves, whereas ‘ANTHEBENEX’ has larger, long ovate leaves.

I claim:

1. A new and distinct variety of Anthurium plant named ‘ANTHDOSDOH’, substantially as illustrated and described herein.
